(19) United States
(12) Patent Application Publication
(10) Pub. No.: US 2021/0008330 A1
(43) Pub. Date: Jan. 14, 2021

(54) METHODS AND SYSTEMS OF VIRTUAL REALITY (VR) BEHAVIOR MODIFICATION

(71) Applicant: Drexel University, Philadelphia, PA (US)

(72) Inventors: Stephanie M. Manasse, Philadelphia, PA (US); Jichen Zhu, Philadelphia, PA (US); Evan M. Forman, Wynnewood, PA (US); Adrienne S. Juarascio, Philadelphia, PA (US); Robert C. Gray, Seattle, WA (US)

(21) Appl. No.: 16/924,592

(22) Filed: Jul. 9, 2020

Related U.S. Application Data
(60) Provisional application No. 62/871,921, filed on Jul. 9, 2019.

(51) Int. Cl. A61M 21/00; G06F 3/01; G09B 19/00; G09B 5/02

(52) U.S. Cl. CPC A61M 21/00; G06F 3/011; A61M 2209/088; G09B 5/02; A61M 2021/005; G09B 19/00

(57) ABSTRACT

One aspect of the invention provides a system for virtual reality (VR) behavior modification. The system includes a VR headset adapted or configured to generate a VR environment comprising at least one VR object corresponding to a behavioral condition of a user; display the VR environment to the user; receive a response from the user comprising an interaction of the user with the at least one VR object; determine a behavioral modification score for the user based on the received response; and alter the at least one VR object based on the received response and the determined behavioral modification score.

### CROSS-REFERENCE TO RELATED APPLICATION(S)

This application claims the benefit of priority of U.S. Provisional Patent Application Ser. No. 62/871,921, filed Jul. 9, 2019. The entire content of this application is hereby incorporated by reference herein.

### BACKGROUND OF THE INVENTION

Binge eating (consuming unusually large amounts of food within a discrete time period, while experiencing a sense of loss of control) and its severe manifestation, binge eating disorder (BED) are debilitating conditions that affect 12.5 million Americans (and 133 million worldwide). Over 45 million people will experience clinically significant binge eating in their lifetime. Binge eating is associated with depression, impaired work performance, decreased social functioning, reduced quality of life, obesity, and resulting type 2 diabetes and heart disease. Psychiatric treatment costs range from $6,000 per year for outpatient therapy to $30,000 for a month of residential treatment. In addition, many patients are prescribed psychiatric medication which adds $3,000 per patient per year. Moreover, medical treatment for associated conditions (e.g., obesity and diabetes) adds additional costs of over $215 billion ($3,500 per patient per year).

### SUMMARY OF THE INVENTION

One aspect of the invention provides a system for virtual reality (VR) behavior modification. The system includes a VR headset adapted or configured to generate a VR environment comprising at least one VR object corresponding to a behavioral condition of a user; display the VR environment to the user; receive a response from the user comprising an interaction of the user with the at least one VR object; determine a behavioral modification score for the user based on the received response; and alter the at least one VR object based on the received response and the determined behavioral modification score.

This aspect of the invention can have a variety of embodiments. The behavioral condition can be binge eating or binge eating disorder. The at least one VR object can include a graphical depiction of food.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 is a graph depicting changes in binge episodes over two weeks in a controlled study in which some users utilized an embodiment of the invention.

FIG. 2 is a photograph of a user utilizing an OCULUS® VR system according to an embodiment of the invention.

FIGS. 3A-3E are a series of screen captures from a VR environment according to an embodiment of the invention. The hands correspond to the user's manipulation of the controllers depicted in FIG. 2.

FIG. 4 is a graph of binge episodes over two weeks in a study in which users utilized an embodiment of the invention.

FIG. 5 is a flow chart depicting a method according to an embodiment of the invention.

### DEFINITIONS

The instant invention is most clearly understood with reference to the following definitions.

As used herein, the singular form “a,” “an,” and “the” include plural references unless the context clearly dictates otherwise.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

### DETAILED DESCRIPTION OF THE INVENTION

### Unmet Clinical Need and Current Standard of Care

### Limitations in the Current Standard of Care

Cognitive behavioral therapy (CBT) is considered the first-line treatment for binge eating yet up to 70% of patients remain fully or partially symptomatic at post-treatment, suggesting a significant need to improve outcomes. A second major limitation in the current standard of care is the fact that CBT is often intensive and expensive, thus necessitating that treatments be made more efficient and disseminable.

Standard Treatments Likely Fail Because they do not Adequately Target Inhibitory Control

Inhibitory control, the cognitive capacity (centered in the prefrontal cortex) that enables withholding automatic responses to rewarding stimuli (e.g., food) has especially strong theoretical and empirical links to binge eating. In-person explicit weekly talk therapies are unable to provide the highly specific, direct, repeated practice necessary for improving inhibitory control. In fact, an influential review of cognitive trainings stated that sustained improvements in cognitive capacities can only occur when training is direct (i.e., specifically trains a cognitive capacity), highly specific (i.e., does not mix any other strategies), and repeated over many trials (e.g., hundreds of trials over weeks).

### Virtual Reality Confers Many Important Advantages to Traditional Serious Games

First, the more realistic imagery of VR better approximates encountering palatable foods in the natural environment. In samples of individuals with eating disorders (EDs), exposure to real food and VR food stimuli produce equivalently high physiological stress responses and exposure to 3D images, compared to 2D images, induces greater desire to consume the food. Secondly, a VR ICT requires inhibition of realistic motor movements, which may increase the potential for transfer to eating behavior. Thirdly, VR induces a “sense of presence”, i.e., a feeling of being immersed, which is likely to result in greater adherence with trainings.

### Stakeholder Analysis and Value Proposition

The first major stakeholder involves the 2.5 million people who receive outpatient treatment, from which the vast majority do not receive adequate benefit. Thus, our technology can serve as an enhancement to traditional treatment. We can demonstrate value by improving symptoms and reducing subsequent spending. Clinicians, patients and insurers would be willing to pay to speed recovery, reduce symptoms, decrease the need for medication and prevent the need for more intensive (e.g., residential) treatment. The second major stakeholder is the group of 10 million untreated patients. For these patients, our technology may be the only therapeutic alternative available to them. For these individuals, we can demonstrate value by reducing binge eating; reducing symptoms of anxiety, depression, and psychological distress; and improving social and work functioning, and control of weight, glucose, and blood pressure—all of which would work to reduce healthcare costs. We believe this need will remain stable into the foreseeable future. Most BED patients will be covered by managed care, and few BED-specialty clinicians participate in HMO panels. Finally, the data generated from this technology are of great interest to stakeholders. Mobile health consumers are willing to provide vast quantities of data, if use of these data result in improvements in their health and in understanding of their illness. Many also are willing to consent to their data being shared, and these data have robust market value to researchers, insurers, and marketers.

### Envisioned Product

One aspect of the invention provides a personalizable, virtual reality “serious game” ICT (TakeControlVR) for reducing binge eating. TakeControlVR is highly disseminable as it can be used regularly, at home, by users either as a stand-alone intervention or in conjunction with in-person CBT. TakeControlVR trains individuals with binge eating to behaviorally inhibit their automatic approach responses to highly palatable food. TakeControlVR will meet a critical unmet need (i.e., enhancement of inhibitory control) in the treatment of binge eating. Additionally, the envisioned product will allow for personalization such that (1) the training adapts to the users' ability so that improvement can continue over time, and (2) the training is personalized such that it trains inhibition to patients' specific binge trigger foods. The TakeControlVR virtual space takes place in a kitchen and gamification allows for users to move up “levels” at increasing difficulties. In order to enhance long-term usage, proposed enhancements to TakeControlVR include creating several virtual environments (e.g., restaurants, social situations, dining room), increasing personalization capabilities (e.g., creating and utilizing a wider range of foods) and including more gamification features (e.g., obtaining badges, points).

### Method and System of Virtual Reality (VR) Behavior Modification

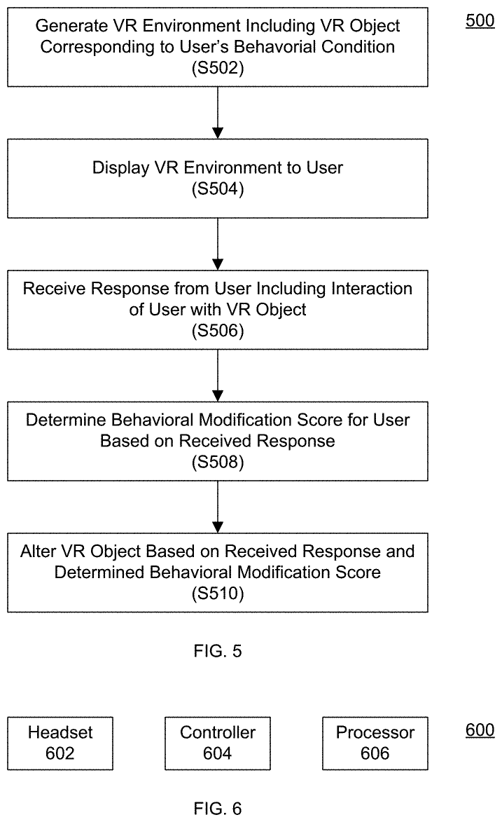

Referring now to FIGS. 5 and 6, aspects of the invention provide a method 500 and system 600 for virtual reality (VR) behavior modification.

Referring now to FIG. 6, the system 600 can include a VR headset 602 such as those available under the OCULUS® trademark from Facebook Technologies, LLC of Menlo Park, Calif.

The system 600 can also include one or more controllers 604 that track the user's hands. Suitable controllers 604 are also available under the OCULUS® trademark from Facebook Technologies, LLC of Menlo Park, Calif. and the NINTENDO® WII® console available from Nintendo of America Inc. of Redmond, Wash. In other embodiments, the controller 604 uses a camera to detect movement of the user (e.g., the user's hands). Suitable controllers 604 are available under the KINECT® trademark from Microsoft Corporation of Redmond, Wash.

The system 600 can include one or more processors 606 adapted and configured to implement one or more methods including those described herein. The processors 606 can be integrated within headset 602 and/or controller 604 or can be physically separate from headset 602 and/or controller 604 and communicatively coupled through a wired or wireless link, whether locally or over a network such as the internet.

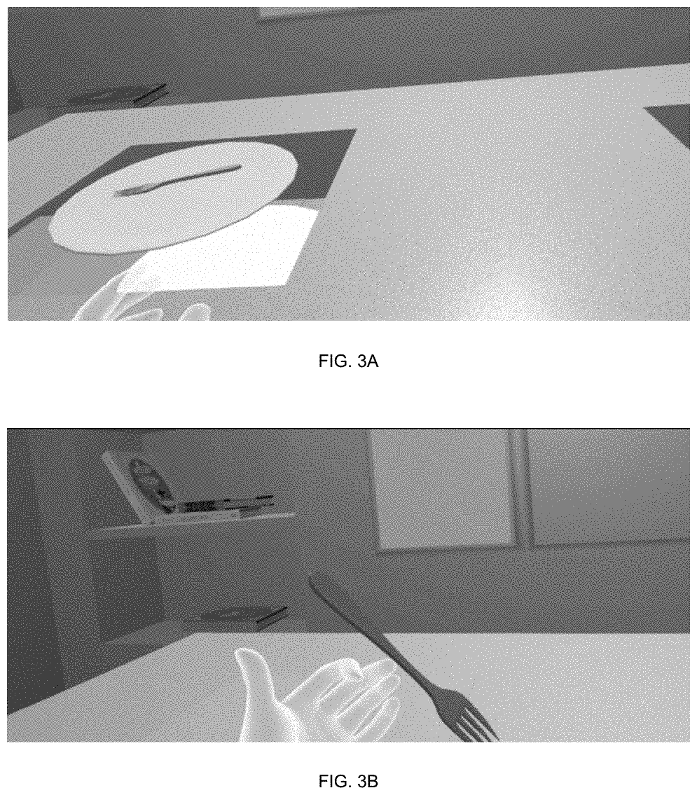

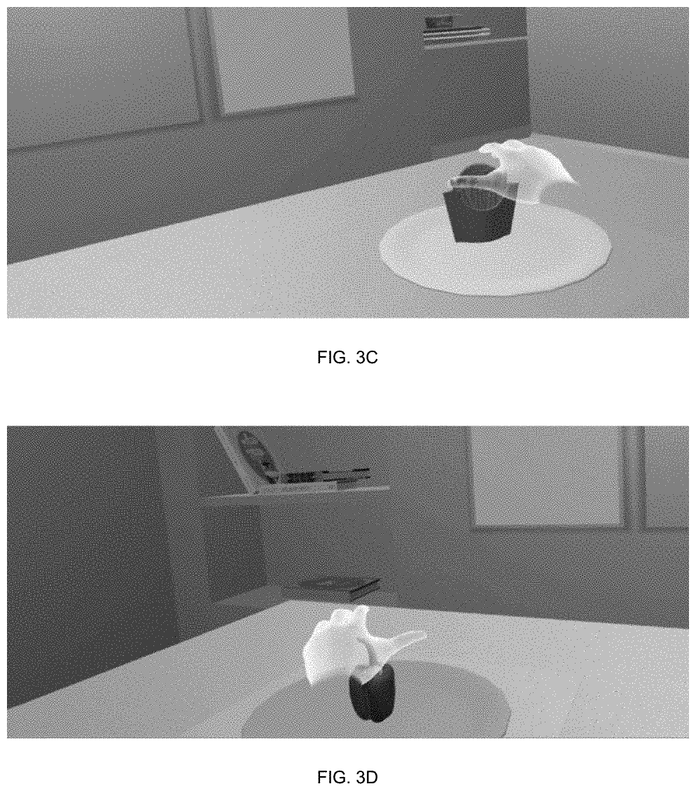

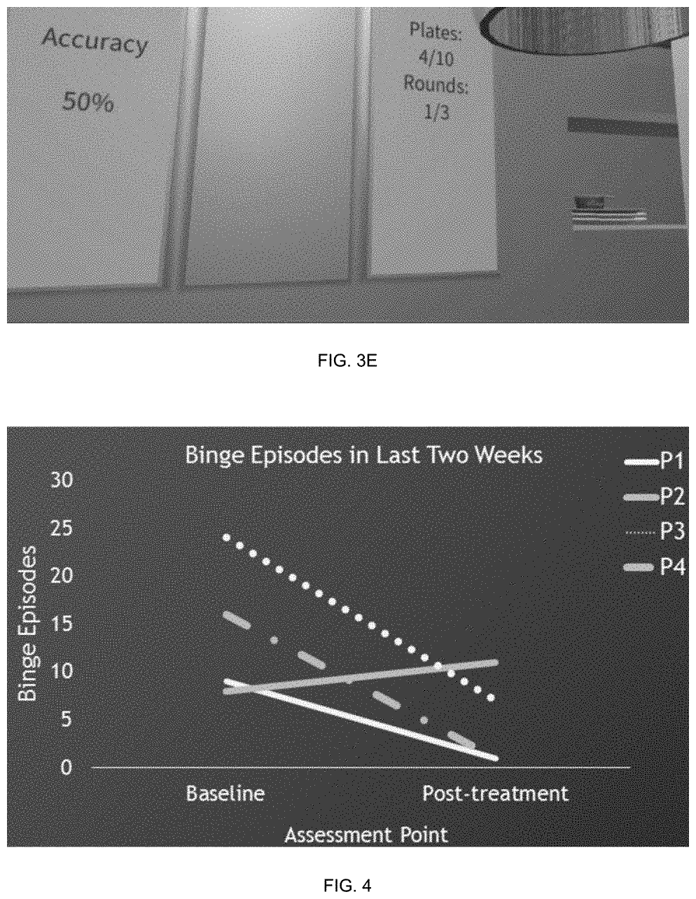

Referring to FIG. 5, in step S502, the processor can generate a VR environment including a VR object corresponding to the user's behavioral condition. For example, if the user's behavioral condition is binge eating or binge eating disorder, the at least one VR object can include graphical depiction(s) of food or food-related items. For example and referring to FIGS. 3A-3E, embodiments of the invention can present depictions of plates (FIGS. 3A, 3C, and 3D), utensils (FIG. 3B), and food (French fries in FIG. 3C and vegetables in FIG. 3D).

In step S504, the VR environment is displayed to the user (e.g., via the VR headset).

In step S506, an interaction of the user with the at least one VR object is received. The response can be captured and/or transmitted using controller 504. The interaction can include grasping or a similar motion or the absence of an grasping (e.g., refraining from interacting with the object). In some embodiments, the interaction is displayed in real time in the VR environment. For example, a representation of a hand is depicted in FIGS. 3A-3D.

In step S508, a behavioral modification score is calculated. For example, the behavioral modification score can reflect a percentage of food or unhealthy food from which the user abstained. FIG. 3E shows the display of a behavioral modification score within the VR environment.

In STEP S510, at least one VR object is altered based on the received response and the determined behavioral modification score. For example, the displayed VR objects can become more or less tempting to the user based on the behavioral modification score.

### Working Example

Applicant developed a working prototype of TakeControlVR. Since January 2019, Applicant recruited 10 participants (7 completed, 3 in-progress) with binge eating (n=5 randomly assigned take home and TakeControlVR for two weeks, and n=5 assigned to use a non-VR version at home for two weeks).

As demonstrated in the table, participants who received TakeControlVR rated the training as easy to use, perceived it as effective, and noted that they would continue to use the training if given the opportunity (see Table, Right).

TABLE 1

| Satisfaction/acceptability item | Mean rating out of 5 (SD)* |
|---|---|
| Easy to use | 4.25 (.50) |
| Had fun while using | 4.25 (.96) |
| Looked forward to using | 4.50 (1.00) |
| Became part of daily routine | 4.50 (1.00) |
| Would continue using | 4.50 (1.00) |
| Training was helpful to reduce binge eating | 4.00 (1.00) |
| Was like resisting impulses in everyday life | 4.00 (1.00) |

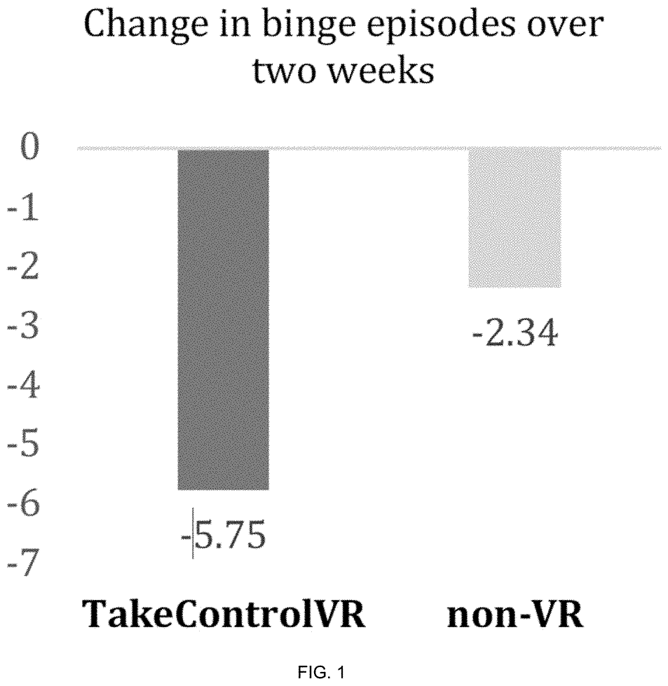

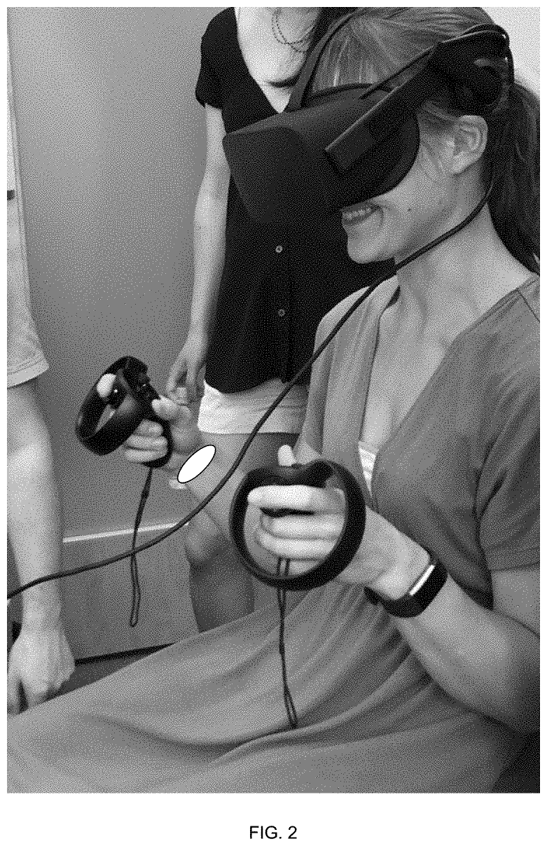

No participants reported any difficulties with nausea or dizziness with VR. Notably, participants reported that completing the trainings felt like resisting impulses in everyday life. Additionally, participants perceived the training to be helpful, which was also demonstrated by large decreases in binge eating experienced by 4/5 participants. Furthermore, these decreases in binge eating with TakeControlVR were greater than those in the non-VR version (see FIG. 1), demonstrating early signs that a VR version of the training confers additional benefits. This advantage of 3.5 binge episodes represents a clinically significant difference (over 11 binge episodes over 1 month and 22 over 2 months) from the non-VR group. Additionally, 100% of TakeControlVR participants reported that they would consider purchasing TakeControlVR if available. By contrast, only 1/3 non-VR participants reported that they would purchase the non-VR version, demonstrating the significantly increased enthusiasm for a VR (versus non-VR) ICT.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A system for virtual reality (VR) behavior modification, the system comprising:
a VR headset adapted or configured to:
generate a VR environment comprising at least one VR object corresponding to a behavioral condition of a user;
display the VR environment to the user;
receive a response from the user comprising an interaction of the user with the at least one VR object;
determine a behavioral modification score for the user based on the received response; and
alter the at least one VR object based on the received response and the determined behavioral modification score.

2. The system of claim 1, wherein the behavioral condition is binge eating or binge eating disorder.

3. The system of claim 1, wherein the at least one VR object comprises a graphical depiction of food.
